(19) United States
(12) Patent Application Publication
(10) Pub. No.: US 2019/0000027 A1
(43) Pub. Date: Jan. 3, 2019

(54) HELIOTROPIUM.ARBORESCENS PLANT WITH A LARGER NUMBER OF PETALS AND A METHOD FOR PRODUCING THE HELIOTROPIUM PLANT

(71) Applicant: Koichiro Nishikawa, Kanagawa (JP)

(72) Inventor: Koichiro Nishikawa, Kanagawa (JP)

(21) Appl. No.: 16/121,748

(22) Filed: Sep. 5, 2018

Related U.S. Application Data
(63) Continuation-in-part of application No. PCT/JP2017/029240, filed on Aug. 7, 2017.

(30) Foreign Application Priority Data
Mar. 6, 2017 (JP) PCT/JP2017/009854

(51) Int. Cl. A01H 5/02; A01H 4/00; A01H 6/00

(52) U.S. Cl. CPC A01H 5/02; A01H 1/02; A01H 6/00; A01H 4/008

(57) ABSTRACT

The Heliotropium.arborescens plant (1) of the present invention comprises at least one flower with more than 6 petals. The number of petals consists of the number of the outer petals (1a) and the inner petaloids (1b) of the Heliotropium. The Heliotropium.arborescens plant (1) has a double-flower trait with many petals, therefore, it is more valuable.

### BACKGROUND OF THE INVENTION

### 1. Field of the Invention

The present invention relates to a Heliotropium.arborescens plant which has a unique number of petals and (a) method for producing the Heliotropium plant.

### 2. Description of the Related Art

Heliotropium belongs to the family Boraginaceae, the genes Heliotropium, and 250 varieties prevail widely in some tropical and temperate zones. A few varieties among them are able to be used as garden plants. The name of the genus is derived from the Greek words helios (sun) and trope (rotation), because it was once believed that the flowers rotated together with the movement of the sun.

Heliotropium.arborescens (also known as its Japanese names Heliotropu or Nioi-murasaki), which is shown in FIG. 18, is native to Peru in South America and planted for a long time. This Heliotropium.arborescens has 50 to 100 cm in height, the funnel-shaped flowers have purple or white coloration, the diameter of the flowers is about 3 mm, the small flowers are crowded in a dome-shape and form cymes. In addition, this Heliotropium.arborescens is known as the first perfume material ever created in the world which produces essential oil and emits a sweet smell similar to vanilla.

Heliotropium.europaeum (also known as its Japanese name: Western breed Kidatirurisou) is one of Heliotropium varieties and an annual plant. Heliotropium.europaeum is native to South Europe. The height of Heliotropium.europaeum reaches 40 to 50 cm and blooms in summer. This Heliotropium.europaeum is also nicknamed as Big Heliotropium, because it creates big inflorescences. The flower's diameter is about 4 mm, and its color of the flowers is either blue or white. Also the smell of the flowers is very faint (refer to Non-patent Reference 1 “Loretta Barnard and co-authors, FLORA (Volume One), Publication in 2003 by Gordon Cheers, p. 689-690”, and Non-patent Reference 2 “Ko Aoba and co-authors, Encyclopedia of Gardening Plants 4, Publication in October, 1990 by Shougakukan, p. 373”).

The above-mentioned Heliotropium is weak to cold, and Heliotropium.arborescens can pass the winter in the west from the Kanto region where the climate is mild, and the stems become woody and shrubby. Every variety is weak to dry condition. The leaves become black and have winkles without water, therefore, planting Heliotropium requires a lot of attention.

Conventionally, flowers that are more voluminous than a single flower, such as double flowers, with numerous petals look elegant and tend to enhance its commercial value since they are more attractive to consumers, even among plants of the same cultivar. For example, the inventions that increase the number of petals such as Verbena (EP 0995354 A2) and Calibrachoa (U.S. Pat. No. 7,786,342 B2) have succeeded in enhancing the value of each flower. Further, as shown in FIG. 19, Petunia with double flowers has recently started to be sold, and the value of the Petunia is increasing, because it has more volume than a single flower.

### BRIEF SUMMARY OF THE INVENTION

However, the first problem with conventional Heliotropium.arborescens is that it has relatively small number of petals so that the flowers look less voluminous. The conventional Heliotropium.arborescens has gamopetalous flowers and the number of the flower petals is only five. In addition, the diameter of the flower is about 3 mm, which is very small, so that the flowers look less voluminous.

In other words, the flower of the conventional Heliotropium.arborescens comprises always five petals, and there is no Heliotropium.arborescens with double flowers, which have a commercial value. Furthermore, the flower size is small so that it damages the impression of the flower and its commercial value. Therefore, there is no doubt that creating a new Heliotropium.arborescens with more petals and double flowers can enhance its commercial value. In addition to this, if a new Heliotropium.arborescens having larger size of the flower compared to the conventional variety is created, it is obvious that the commercial value would be very high.

The present invention has been made in view of the abovementioned problems, and has an object to provide a Heliotropium.arborescens plant with a larger number of petals than conventional cultivars of Heliotropium, and a method for producing the Heliotropium.arborescens plant. Furthermore, in addition to having a feature such that its flower has a larger number of petals, the present invention also aims to provide a new cultivar of Heliotropium.arborescens with a larger size of the flower.

### DISCLOSURE OF INVENTION

In order to solve the aforementioned issues, the present invention is a Heliotropium.arborescens plant comprising at least one flower with more than 6 petals, wherein the Heliotropium.arborescens plant comprises a multiple petal gene, as found in Heliotropium.arborescens variety “HDF-1”, representative tissue culture having been deposited with the international depositary authority under the deposit number FERM BP-22339.

In this Heliotropium.arborescens plant, preferably, wherein the flower includes 7 to 10 petals.

In this Heliotropium.arborescens plant, preferably, wherein the flower includes 11 to 13 petals.

In this Heliotropium.arborescens plant, preferably, wherein the flower includes more than 14 petals.

In this Heliotropium.arborescens plant, preferably, wherein the flower of the Heliotropium plant is a double-flower type with outer petals as a gamopetalous flower, and inner petaloids, which are separated with each other, near the center of the flower, and wherein “the number of petals” consists of the number of the outer petals and the inner petaloids of the Heliotropium.

In this Heliotropium.arborescens plant, preferably, wherein the number of the inner petaloids is more than 1 petal.

In this Heliotropium.arborescens plant, preferably, wherein the number of the inner petaloids is more than 4 petals.

In this Heliotropium.arborescens plant, preferably, wherein the number of the outer petals is more than 7 petals.

In this Heliotropium.arborescens plant, preferably, wherein the size of the separated inner petaloids is more than half of the size of the outer petals.

In this Heliotropium.arborescens plant, preferably, wherein the size of the separated inner petaloids is more than ⅔ of the size of the outer petals.

In this Heliotropium.arborescens plant, preferably, wherein the Heliotropium.arborescens plant comprises no pollen.

In this Heliotropium.arborescens plant, preferably, wherein the Heliotropium.arborescens plant comprises no stamen.

In this Heliotropium.arborescens plant, preferably, wherein the Heliotropium.arborescens plant is produced from the tissue culture having the deposit number FERM BP-22339, which is the variety “HDF-1”.

In order to solve the aforementioned issues, the present invention is seed of the said Heliotropium.arborescens.

In order to solve the aforementioned issues, the present invention is a tissue culture of cells produced from the said plant, wherein said cells of the tissue culture are produced from a plant part selected from the group consisting of seed, leaf, pollen, embryo, cotyledon, hypocotyl, meristematic cell, root, root tip, pistil, anther, flower, stem, and petiole.

In order to solve the aforementioned issues, the present invention is a method of producing the said Heliotropium.arborescens plant comprising; (a) crosssing, as a male or female parent, the Heliotropium.arborescens plant of claim 1 that has more than 6 petals, with a Heliotropium.arborescens plant that does not have more than 6 petals, (b) selecting progeny that has more than 6 petals.

In order to solve the aforementioned issues, the present invention is a Heliotropium.arborescens plant obtained by utilizing either one of pollen, ovum, cell, and genetic information relating to DNA and RNA of the abovementioned Heliotropium.arborescens plant.

In order to solve the aforementioned issues, the present invention is a method for producing a Heliotropium.arborescens plant comprising; (a) utilizing either one of pollen, ovum, cell, and genetic information relating to DNA and RNA of the Heliotropium.arborescens plant of claimed above so as to produce the Heliotropium.arborescens plant.

According to the present invention, the Heliotropium.arborescens plant has more than 6 petals. Therefore, the Heliotropium.arborescens plant has the characteristic of having more number of petals as compared to conventional cultivars of Heliotropium.arborescens, and the plant has more commercial value.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention will be described hereinafter with reference to the annexed drawing. It is to be noted that the drawing is shown for the purpose of illustrating the technical concepts of the present invention or embodiments thereof, wherein:

FIG. 1 shows Heliotropium.arborescens plants in bloom according to Embodiment 1 of the present invention.

FIG. 2 shows Heliotropium.arborescens plants in bloom according to Embodiment 1 of the present invention.

FIG. 3 shows the flower of Heliotropium.arborescens according to Embodiment 1 of the present invention.

FIG. 4 shows Heliotropium.arborescens plants in bloom according to Embodiment 1 of the present invention.

FIG. 5 shows a flowchart to show the processes of cross breeding.

FIG. 6 shows the Heliotropium.arborescens plant comprising a flower with more than 12 petals according to Embodiment 1 of the present invention.

FIG. 7 shows a photograph of when the Heliotropium.arborescens flower with more than 14 petals are separated.

FIG. 8 shows a photograph of a double-flower type Heliotropium.arborescens plant.

FIG. 9 shows the flower of Heliotropium.arborescens according to Embodiment 1 of the present invention.

FIGS. 10a to 10d show photographs of when the Heliotropium.arborescens flower are separated.

FIGS. 11a to 11d show photographs of when the Heliotropium.arborescens flower are separated.

FIG. 12a shows a photograph of Heliotropium plant having more than 4 inner petaloids and the color of the flowers is purple.

FIG. 12b shows a photograph of Heliotropium plant having more than 2 inner petaloids and the color of the flowers is white.

FIG. 13 shows a photograph of Heliotropium.arborescens flower according to Embodiment 2 of the present invention.

FIG. 14 shows a photograph of the Heliotropium.arborescens flower according to Embodiment 2 of the present invention.

FIG. 15 shows a photograph of the Heliotropium.arborescens flower according to Embodiment 2 of the present invention.

FIGS. 16a to 16d show photographs of the Heliotropium.arborescens whose flower diameter is more than 15 mm and the number of the petals is 5.

FIGS. 17a to 17b show photographs of the Heliotropium.arborescens whose flower diameter is more than 15 mm and the number of the petals is 5.

FIGS. 18a to 18c show photographs of conventional Heliotropium.arborescens plants which are in bloom.

FIGS. 19a to 19d show photographs of Petunia plants which are in bloom.

### DETAILED DESCRIPTION OF THE INVENTION

Embodiments of the present invention, as best mode for carrying out the invention, will be described hereinafter with reference to the drawing. It is to be understood that the Embodiments described herein are not intended as limiting, or encompassing the entire scope of, the present invention.

### Embodiment 1

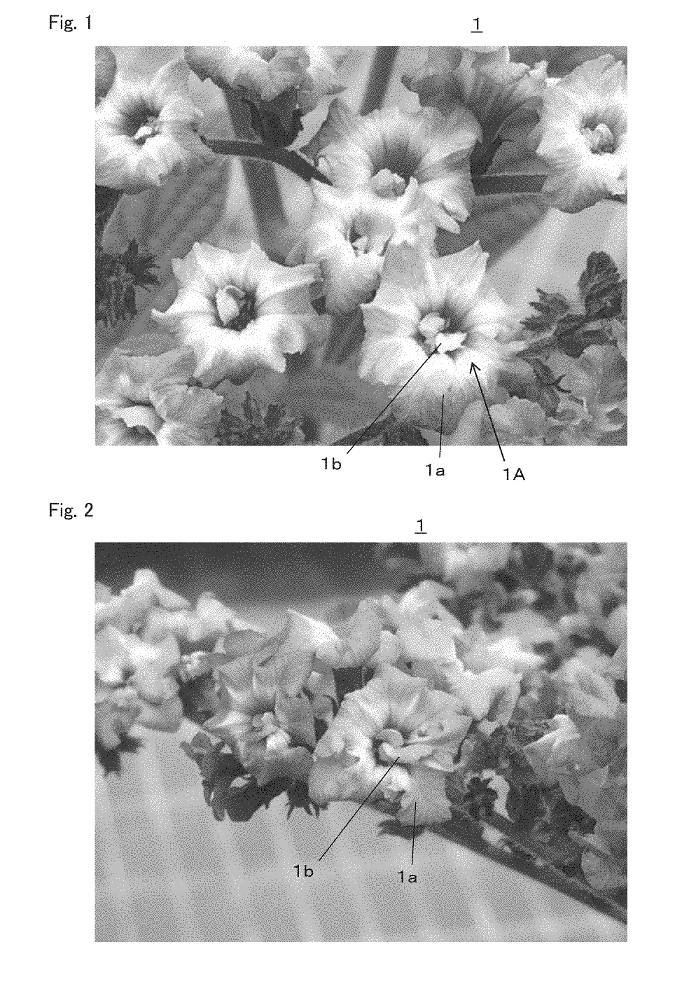

Referring from FIG. 1 to FIG. 11, a Heliotropium.arborescens plant according to Embodiment 1 of the present invention will be described. Hereinafter, the variety of Heliotropium in this Embodiment 1 is Heliotropium.arborescens. It is noted that Heliotropium.arborescens is different from the variety called Heliotropium.europaeum because sometimes these two varieties are confused with each other.

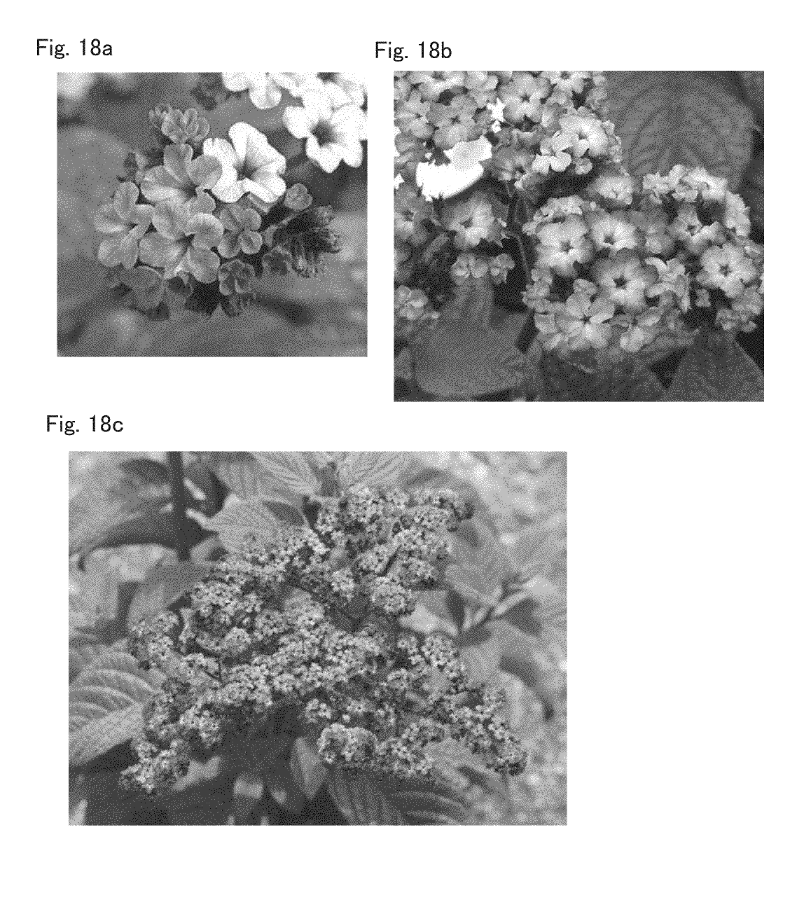

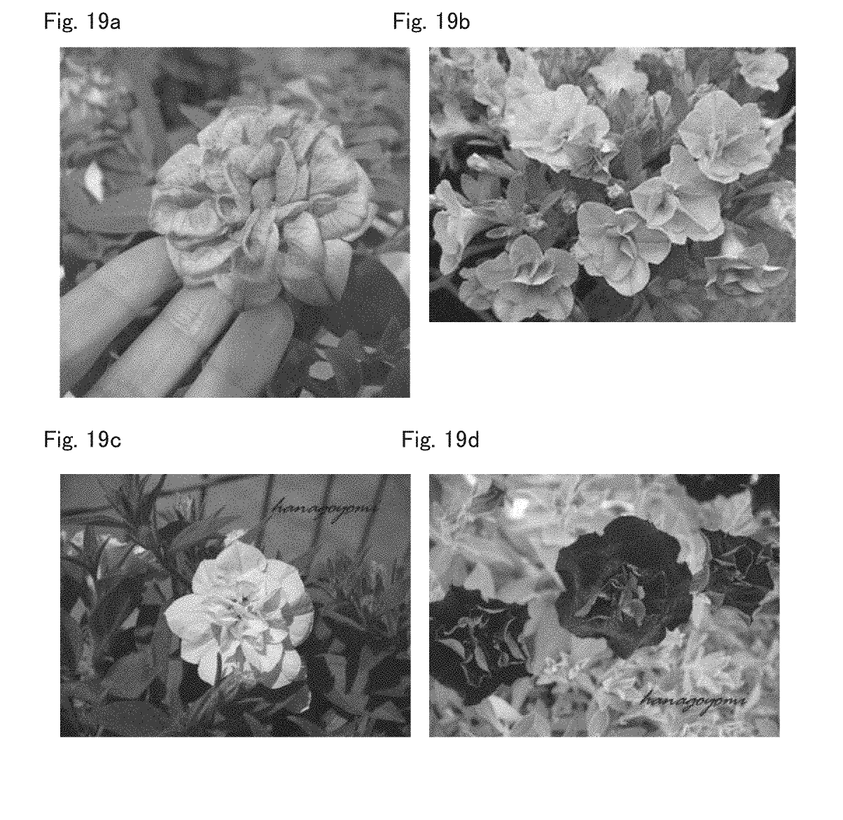

The inventor of the present application has successfully obtained a plant cultivar shown in FIG. 1 to FIG. 4 (hereinafter referred as “Heliotropium 1”), having a characteristic of double flowers, after repeating the process of seeding, crossing and selection. The number of petals of Heliotropium 1 is larger (e.g. more than 6 petals) than conventional Heliotropium. On the other hand, all flowers of the conventional cultivars are characterized by having 5 petals (gamopetalous corollas, which are disected into 5 petals), such as being shown in FIG. 18, and there has been no Heliotropium variety having more than 6 petals ever in history.

Heliotropium 1, shown in FIG. 1 to FIG. 4, has not only the outer petals 1a as the gamopetalous flowers, but also the separated petaloids 1b in the inner side of the outer petals 1a, so that Heliotropium 1 shows double flowers. Herein, the term “double flowers” means that a plurality of inner petaloids 1b is arranged inside the outer petals 1a of the gamopetalous flowers so that Heliotropium 1 looks like it has many outer petals 1a and many inner petaloids 1b. Although Heliotropium 1 has white or blue colored flowers, sometimes it shows red-purple colored flowers. The flower coloration of Heliotropium 1 is not limited.

Heliotropium 1 according to Embodiment 1 comprises at least one flower with more than 6 petals, preferably 7 to 10 petals, more preferably 11 to 13 petals, and even more preferably more than 14 petals. Herein, the number of the outer petals 1a of the gamopetalous flowers is more than 7. The number of the inner petaloids 1b is more than 1, and preferably more than 4. For example, it is observed that Heliotropium 1A in FIG. 1 comprises more than 7 outer petals 1a and the number of the separated inner petaloids 1b is 3. The term “the number of petals” herein consists of outer petals (1a) and inner petaloids (1b) around pistil of the Heliotropium 1. In other words, “the number of petals” includes the number of staminodes (stamens converted into petals) and inner petaloids, which have grown up and become large outer petals, around the pistil.

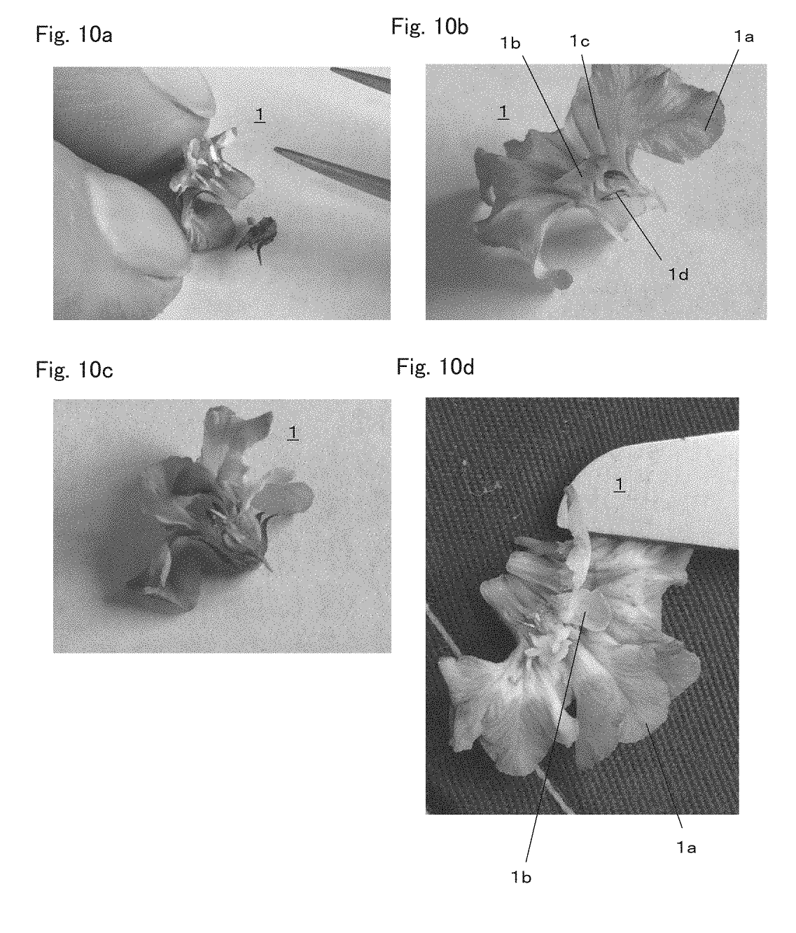

The flower of Heliotropium belongs to Gamopetalae academically, and the petals are fused in the base region so that the number of the petal might be counted as one. However, in terms of Gamopetalae, the number of the petals is counted according to the number of the fused petals. Specifically, the petal of Gamopetalae is dissected into some lobes in its apex region, the number of the lobes is counted as the number of the petals. Therefore, the number of the lobes is equivalent to the number of the petals. Furthermore, if it is not easy to distinguish the dissected part, the number of the reticulates 1c (refer to FIG. 7, FIG. 10 (b), and etc), which extend in up and down direction, can be counted as the number of the petals, because the reticulates 1c are easy to observe. On the other hand, it is known that the petals of a choripetalous flower are separated in the base region.

Herein, the tussue culture of the Heliotropium plant has been deposited in the following international depositary authority under deposit number FERM BP-22339. Meri-clone organism is included int the tissue culture. The Heliotropium plant according to Embodiment 1 of the present invention also includes a progeny of the Heliotropium plant, a crossbreeding variety (a hybrid plant), and a progeny of the hybrid plant. The information relating to the said deposit will be described as follows.

Kind of deposit: International deposit under the Budapest treaty

Name of the authority: International patent organism depositary, National institute of technology and evaluation

Address: #120, 2-5-8 Kazusakamatari, Kisarazu-shi, Chiba 292-0818, Japan Accession number given by the the international depositary authority: FERM BP-22339

Identification reference given by the depositor: NISHIKAWA2

Date of the original deposit: Jul. 5, 2017

Embodiment 1 of the present invention provides a new Heliotropium plant, which is (a) a cultivar called Variety A, which is produced by obtaining an individual mutant from hybridizing at least one of the individuals selected from Heliotropium.arborescens, and by crossing using the individual mutant and then selecting said cultivar, (b) a cultivar produced by crossing species, wherein at least either one of the parents of the species is selected from the Variety A, or (c) a progeny of one of the cultivars (a) and (b). This crossing includes self-pollination inside an individual plant and cross-pollination between different individual plants. In case of self-pollination, it uses the pistil and pollens collected from an individual plant. In case of cross pollination, one parent must be the Heliotropium plant related to Embodiment 1 of the present invention, while the other parent is not necessary to be such the Heliotropium plant as long as it is possible for the other parent to cross with the Heliotropium plant according to Embodiment 1 and to generate progenies.

A method for producing/breeding the Heliotropium plant according to Embodiment 1 of the present invention has no particular restriction, except that at least either one of the parents should be the Heliotropium plant characterized by having more than 6 petals, preferably 7 to 10 petals, more preferably 11 to 13 petals, and even preferably more than 14 petals. For example, a breeding method for producing the Heliotropium plant comprises; (a) crossing the Heliotropium plant having such traits, and (b) selecting a Heliotropium plant having the traits of the Heliotropium plant from the progenies of the crossed Heliotropium plant. Furthermore, this invention includes a Heliotropium plant (product by process) obtained by a method for producing/breeding the Heliotropium plant, which has the abovementioned characteristics. The method includes every selecting step/crossing step in order to obtain the Heliotropium plant.

Heliotropium can be propagated by using seeds, planting cuttings and layering, both in nature and in deliberate cultivation. The Heliotropium plant according to Embodiment 1 of the present invention can be obtained by utilizing either one of reproduced pollen, ovum, cell, and the genetic information contained in the DNA and RNA of the Heliotropium species with abovementioned characteristics. In other words, any reproducible parts of the Heliotropium plant can be used for reproducing, and the reproduction is not limited to sexual reproduction but also includes asexual reproduction. Herein, the term “plant(s)” includes plant organs, plant tissues, cells, vegetative propagules and the likes, and the plant organs include petals, corolla, flowers, leaves, seeds, fruits, stems, roots, and the likes. And cells of the tissue culture are produced from a Heliotropium plant part selected from the group consisting of seed, leaf, pollen, embryo, cotyledon, hypocotyl, meristematic cell, root, root tip, pistil, anther, flower, stem, and petiole.

The Heliotropium plant according to Embodiment 1 of the present invention has the characteristics as follows. Firstly, the Heliotropium plant is characterized by having more than 6 petals, preferably 7 to 10 petals, more preferably 11 to 13 petals, and even preferably more than 14 petals. In addition to this, the flower of the Heliotropium plant has not only outer petals, but also inner petaloids, which are separated with each other. Note that the aforementioned characteristics or a combination of the characteristics of this new Heliotropium plant is clearly distinguished from the characteristics of conventional Heliotropium species. On the other hand, conventional Heliotropium species are characterized by having 5 petals (academically, gamopetalous corollas, which are dissected into 5 petals). Therefore, it is obvious that the Heliotropium plant 1 having the abovementioned characteristics is a totally new cultivar.

### EXAMPLES

Referring to the examples below, the Heliotropium plant according to Embodiment 1 will be described. It is to be understood that the examples described herein are not intended to limiting the entire scope of the present invention.

### Example 1

The new Heliotropium plant relating to the present invention was invented at a farm located in Okayama Prefecture, Japan, under a breeding program instructed and managed by the inventor. The purpose of this breeding program was to breed a new Heliotropium plant having more petals than conventional Heliotropium species, more specifically, having more than six petals. It is noted that the breeding condition of the new Heliotropium plant according to the present invention is not particularly limited to the condition described in Example 1, as long as the condition is applicable to the Heliotropium plant.

Firstly, the inventor collected 2,000 of hybrid seeds obtained through crossing of cultivars selected from Heliotropium.arborescens owned by the inventor, and bred them in a greenhouse near the inventor's house located at Katsuta-gun in Okayama Prefecture, Japan, with ordinary breeding methods. After crossing the abovementioned seeds with various seeds owned by the inventor for three generations repeatedly, a single mutant individual of a Heliotropium plant with a unique characteristic, which has one extra petal (a separated inner petaloid) around the center of the flower in addition to the five outer ordinal petals, was suddenly obtained.

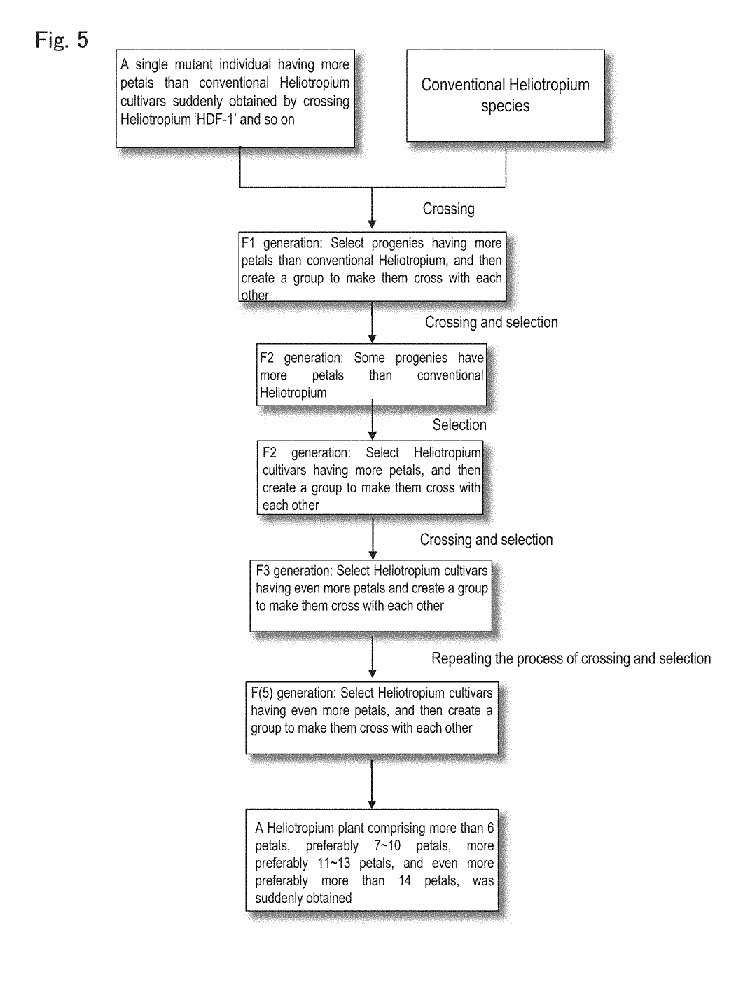

Next, after repeating the crossing and selection processes using the cultivars from a group of progenies obtained from the single mutant individual, a Heliotropium plant having more petals was successfully obtained. To be more specific, as described in FIG. 5, the procedure is as follows: (1) crossing the mutant individual of Heliotropium, which has one extra petal (a separated inner petaloid) around the center of the flower. (2) The Heliotropium plant, which has one or two extra petals (separated inner petaloids) around the center of the flower, was suddenly obtained from a group of F2 generation; (3) next, collecting seeds from the said Heliotropium plant, and selecting Heliotropium cultivars having more petals obtained by breeding the seeds. And crossing the said Heliotropium cultivars, and then selecting Heliotropium cultivars having more petals (repeating the crossing and selection) for multiple generations in order to gradually enhance the characteristic of having more petals. However, it is noted that when Heliotropium cultivars are grown from seeds, the number of the petals is not stable. For example, the ratio of cultivars with more than 6 petals was sometimes below 10% and sometimes over 30% in the next generation. (4) When reaching a stage that some of Heliotropium cultivars have more petals, more specifically, more than 6 petals, preferably 7 to 10 petals, more preferably 11 to 13 petals, and even preferably more than 14 petals, was suddenly emerged. And (5) by repeating crossing and selection of the Heliotropium plant having the said characteristic even further, a Heliotropium plant whose petaloids (separated inner petals) are almost same size as the outer petals was succeeded to obtain. (6) Genetically fixing a desired characteristic by repeating crossing and selection of the Heliotropium plant having the said characteristic even further for multiple generations. In other words, the Heliotropium plant with the desired characteristic can surely be obtained, even in small amounts, by utilizing the crossed Heliotropium plant (as at least either one of the parents for crossing) within multiple generations (which means that the characteristic is repeatable).

As described above, the characteristic of the Heliotropium plant according to Embodiment 1 of the present invention is repeatable/inheritable, which means that the same result can be repeatable if the same breeding method is repeated, and the Heliotropium plant is clearly different from a conventional plant in its characteristic (the number of the petals). In addition, the inventor has already obtained many varieties of Heliotropium plant having this characteristic (having double flowers with many petals) by utilizing the Heliotropium plant, which was initially obtained. This also indicates that the characteristic according to the present invention is repeatable.

### Example 2

Secondly, a crossing experiment was implemented. Specifically, the Heliotropium plant with the specific characteristic herein had been bred and the pollens from stamens of Heliotropium with normal number of petals were deposited into its pistil of the Heliotropium plant. The Heliotropium plant was bred until it generated seeds, and then the seeds collected from the plant were sowed and bred. As a result, the Heliotropium plants comprising more than 6 petals, preferably 7 to 10 petals, more preferably 11 to 13 petals, and even preferably more than 14 petals, were found in the first generation “a child generation (F1)”, the next generation “a grandchild generation (F2)”, and the next next generation (F3) obtained through the said breeding procedures. Herein, the percentages of these Heliotropium plants were sometimes below 10% and sometimes over 30%.

Accordingly, by means of depositing pollens from a fertile commercial cultivar into its pistil of the Heliotropium plant having more than 6 petals according to Embodiment 1 of the present invention, the Heliotropium having the characteristic will be produced in following generations, hence, this shows that this characteristic is inheritable. As described above, in Example 2, the Heliotropium plant with the desired characteristic can surely be obtained by utilizing the Heliotropium plant (at least either one of the parents for crossing) within multiple generations.

### Example 3

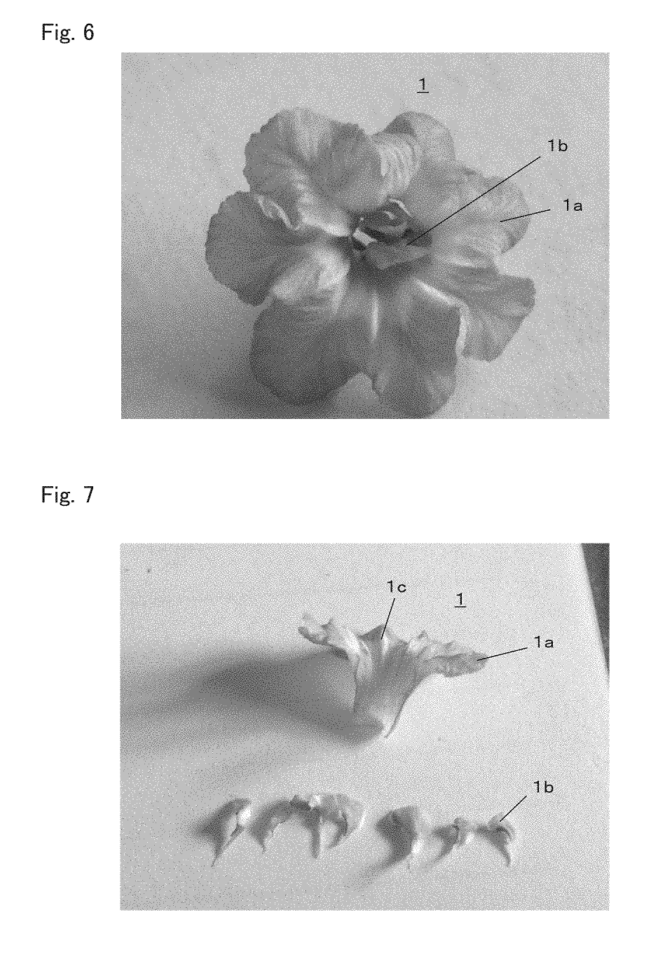

The details of the Heliotropium plant having more than 6 petals will be explained as follows, FIG. 6 shows the Heliotropium plant 1 with 7 outer petals 1a and at least 5 inner petaloids 1b. It is observed that the said Heliotropium has at least 12 petals in total. It is noted that the ratio of outer petals 1a to inner petaloids 1b is not particularly limited to 7:5, and can vary.

### Example 4

FIG. 7 shows the Heliotropium plant 1 with 7 outer petals 1a and 7 inner petaloids 1b. It is observed that the said Heliotropium has 14 petals in total. It is noted that increasing the number of petals means increasing the number of outer petals 1a, increasing the number of inner petaloids 1b, or increasing both the outer petals 1a and inner petaloids 1b.

### Example 5

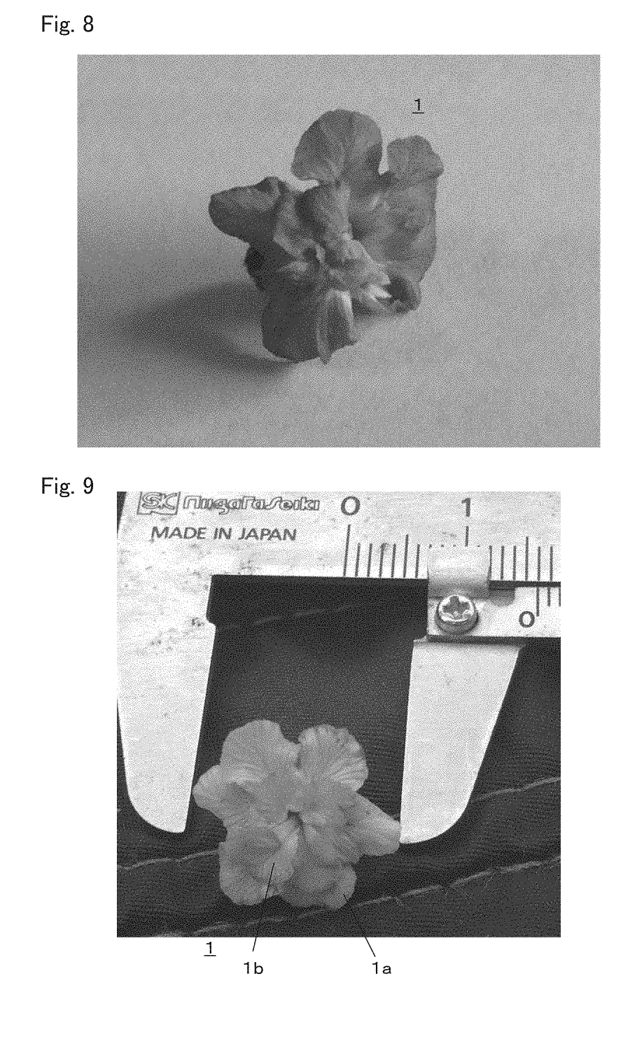

FIG. 8 shows the Heliotropium plant 1 with more petals than before, and the said petals are fused (double) with each other. This is also the case when the Heliotropium plant 1 has flowers with more than 6 petals.

### Example 6

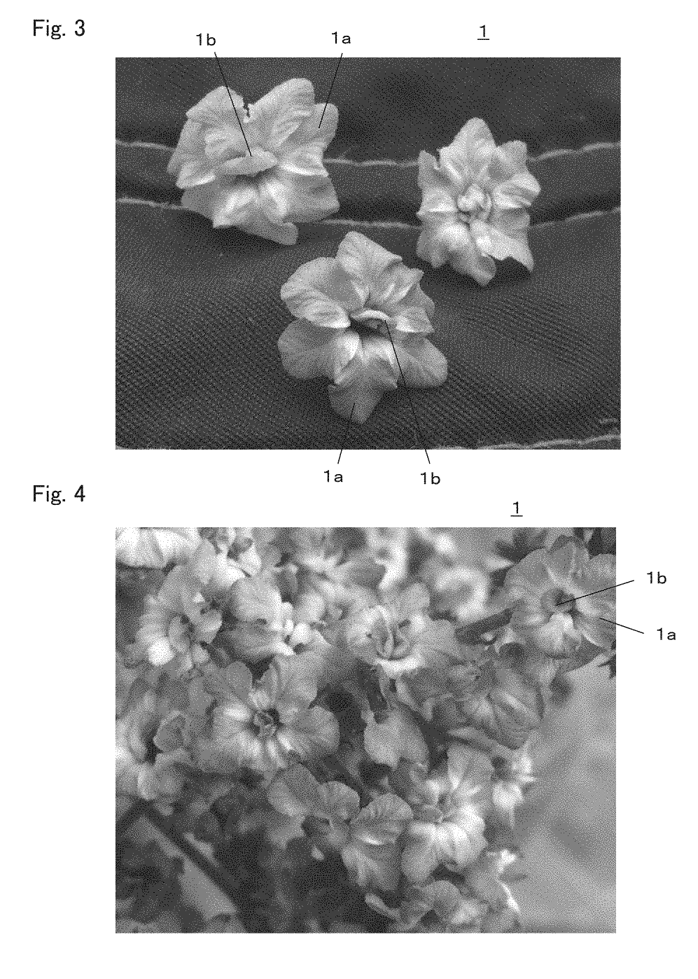

The size (superficial area) of the separated inner petaloids will be explained in Example 6. As mentioned above, a Heliotropium plant, whose petaloids (separated inner petals) are almost the same size as the outer petals, was succeeded to obtain by repeating crossing and selection of the Heliotropium plant. Specifically, it is observed in FIG. 3 and FIG. 9 that the size of the separated inner petaloids 1b is more than half, preferably more than ⅔ of the size of outer petals 1a. This enables the Heliotropium plant 1 with double petals to have more commercial value.

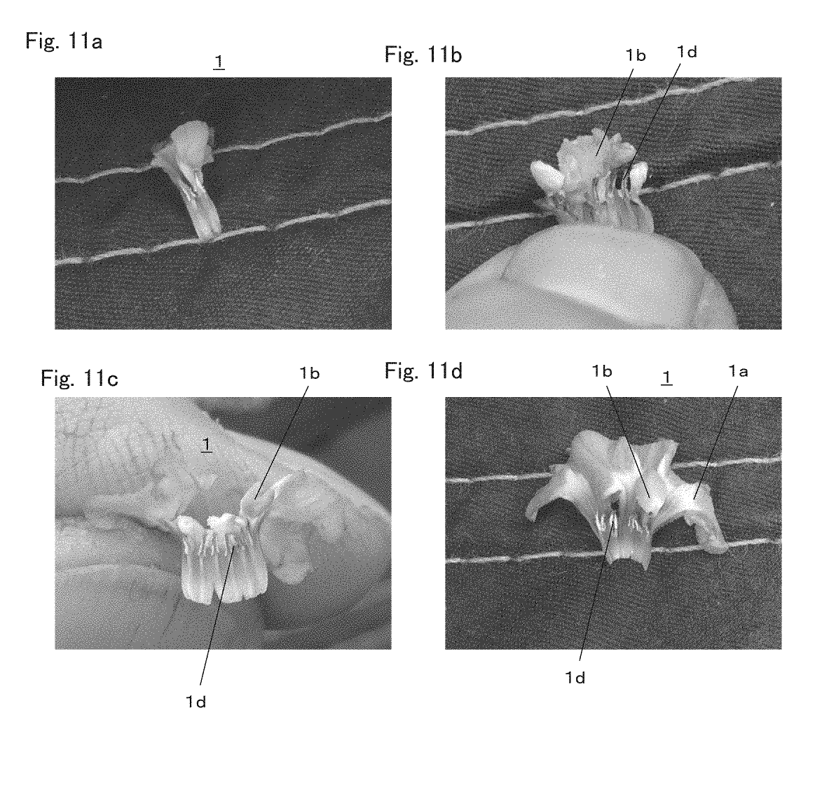

Next, the separated inner petaloids 1b of the Heliotropium plant 1 will be explained by referring to FIG. 10 and FIG. 11. Conventional Heliotropium plants have five stamens, and these stamens do not extend out of the flower. On the other hand, it was observed in FIG. 10 that the stamens were transformed into inner petaloids 1b, and other organs were transformed into inner petaloids 1b. In some cases, all stamens were transformed into inner petaloids 1b.

As described above, the Heliotropium plant according to Embodiment 1 of the present invention comprises more than 6 petals, preferably 7 to 10 petals, more preferably 11 to 13 petals, and even preferably more than 14 petals. The Heliotropium plant comprises the inner petaloids, in addition to the outer petals, so that the Heliotropium plant comprises double-flowers. As a result, the present invention can provide the new Heliotropium plant that is more voluminous and more commercially valuable than conventional Heliotropium. It is to be understood that the Heliotropium plant described herein is not intended to limiting the scope of morphological and ecological characteristics of Heliotropium, except for the number of petals and the size of the flowers.

The present invention is further detailed in the following Examples, which are offered by way of illustration and are not intended to limit the invention in any manner. Standard techniques well known in the art or the techniques specifically described are utilized.

### Example 7

<Breeding History of a Heliotropium Plant “HDF-1 (Deposit No. FERM BP-22339)” with the Claimed Trait>

In 2010, 2000 seeds, which were obtained by crossing Heliotropium.arborescens with each other, were sowed in a controlled greenhouse near the inventor's house located at Katsuta-gun in Okayama Prefecture, Japan, with ordinary breeding methods. After crossing the abovementioned seeds with various seeds owned by the inventor for three generations repeatedly, a single mutant individual (Variety name: NDF-OR1) of a Heliotropium plant with a unique characteristic, which has the usual five outer petals as a gamopetalous flower, and one separated inner petaloid near the center of the flower, was suddenly obtained.

The inventor repeated crossing and selection processes using the cultivars from a group of progenies obtained from the single mutant “NDF-OR1”. As a result of this, a Heliotropium plant having a unique number of the petals was successfully obtained as described in FIG. 5. In order to obtain a Heliotropium plant having more petals, crossing (e.g. a cross between “HDF-OR1” and available varieties like the commercially available Heliotropium arborescens cultivar “Marine”, which is not patented) and selection processes using the cultivars from a group of progenies obtained from the single mutant “HDF-OR1” were conducted repeatedly.

This is traditionally known as crossing and selection processes in the field of breeding. In other words, crossing the Heliotropium cultivars, and then selecting Heliotropium cultivars with more petals (repeating crossing and selection) for multiple generations. As a result, the characteristic of the Heliotropium flower with more petals is gradually enhanced. The gene characterized by having more petals than conventional cultivars can be fixed in the Heliotropium plant by repeating the crossing and selection processes for multiple generations. In other words, the Heliotropium plant with the desired characteristic can surely be obtained, even in small amounts, by utilizing the genetically fixed Heliotropium plant (at least either one of the parents for crossing) within multiple generations, which means that the characteristic is repeatable.

The Heliotropium plant “HDF-1”, in which a desired characteristic is fixed, has been deposited under the Budapest Treaty. The deposit No. is FERM BP-22339. This variety “HDF-1”, which has at least six petals comprising more than five outer petals as a gamopetalous flower and more than one separated inner petaloids near the center of the flower, was obtained. As described above, the Heliotropium flower with larger number of outer petals and inner petaloids has more volume and commercial value. The Heliotropium flower “HDF-1” has a double-flower trait with many petals, therefore, it is more valuable.

### Example 8

### <Botanical Description of HDF-1>

The plant growth habit and botanical description of HDF-1 are very similar to a commercially available Heliotropium arborescens cultivar called “Marine”, which is not patented.

Herein, the number of petals relating to the variety “HDF-1” will be explained. Table 1 shows the comparison of the number of outer petals and inner petaloids between HDF-1 of the present invention and former cultivars. The variety “HDF-1” comprises 6 to 14 petals (quite rarely 15), which are composed of 5 to 14 (quite rarely 15) outer petals and 1 to 7 (quite rarely 8) separated inner petaloids. It is obvious that HDF-1 has more number of petals compared with other cultivars.

It is to be noted that the variety “HDF-1” sometimes comprises the flowers with different number of petals per plant. On the other hand, the conventional Heliotropium varieties, such as “Marine” and “Nagano”, have the flowers with the same number (5) of petals.

TABLE 1

| | The number | The number of | |
|---|---|---|---|
| | of outer | petaloids | |
| | petals | (inner petals) | Total |
| Heliotropium arborescens | 5 to 14 | 1 to 7 | 6 to 14 |
| “HDF-1” of the present | (quite | (quite | (quite |
| invention | rarely 15) | rarely 8) | rarely 15) |
| A conventional Heliotropium | 5 | 0 | 5 |
| variety called “Marine” | | | |
| A conventional Heliotropium | 5 | 0 | 5 |
| variety called “Nagano” | | | |

### Example 9

### <Introgression of a Multiple Petal Gene>

Herein, introgression means the process of transferring a genetic trait (e.g. a multiple petal gene) from one genotype to another. In addition, the Heliotropium variety of the present invention sometimes comprises the flowers with different number of petals. Therefore, most common trait of the flowers (petals) per plant will be mentioned.

A multiple petal gene, which is acquired in the Heliotropium variety “HDF-1”, is independent from other genes such as producing the color of leaves, the color of the flower, the length of the stem, and so on. As a result, this genetic trait can be introgressed from this line into any desired Heliotropium cultivar.

(1) Table 2 shows the crossing result between A as a female and HDF-1 as a male. As a result of this crossing, we obtained Progeny I. And the ratio of Heliotropium plant, which has more than two inner petaloids among F1 generation, was 1.5%.

TABLE 2

| Male parent HDF-1 | Female parent A | Progeny I (F1) |
|---|---|---|
| (1) Heliotropium plant | (1) Heliotropium plant | (1) Heliotropium plant |
| having 5 outer petals, 2 | having 5 outer petals | having 5 outer petals, |
| inner petaloids | (2) Length of nodes | 2 inner petaloids (7 |
| (2) Length of nodes | on a stem: long | petals in total) |
| on a stem: medium | (3) Size of the leaves: | (2) Length of nodes |
| (3) Size of the leaves: | large | on a stem: long |
| medium | | (3) Size of the leaves: |
| | | large |

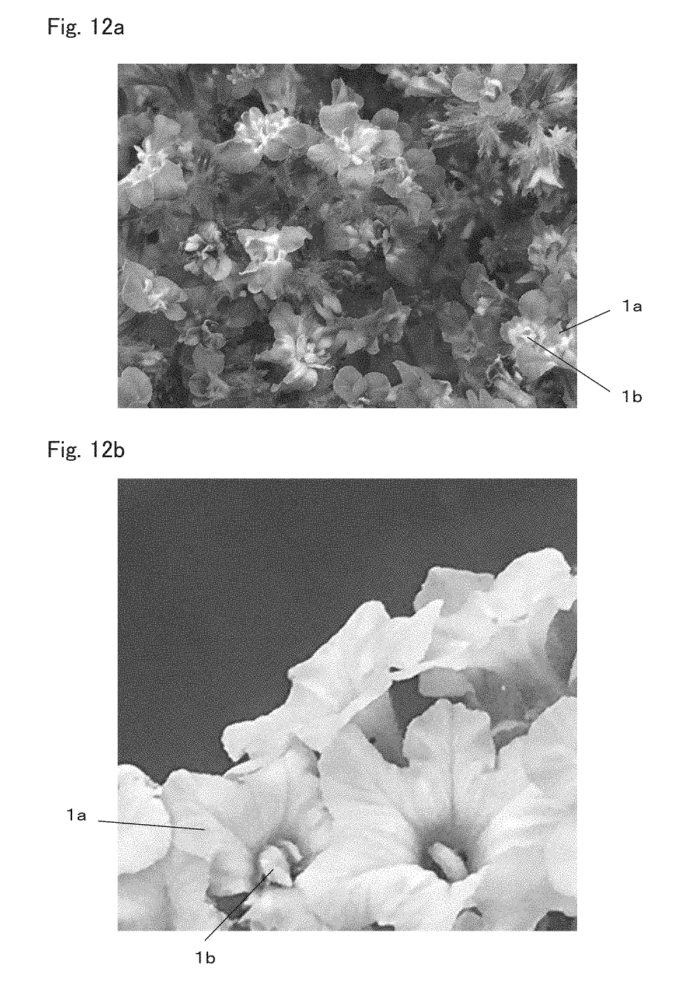

(2) Table 3 shows the crossing result between B as a female and HDF-1 as a male. As a result of this crossing, we obtained Progeny C. And the ratio of Heliotropium plant, which has more than two inner petaloids among F1 generation, was 1%. Herein, FIG. 12A shows that Heliotropium plant having more than 4 inner petaloids 1b and the color of the flowers is purple. FIG. 12B shows that Heliotropium plant having more than 2 inner petaloids 1b and the color of the flowers is white. Furthermore, as shown in Table 4, crossing between Progeny C with each other was conducted. As a result, we obtained Progeny D. And the ratio of Heliotropium plant, which has more than two inner petaloids among Progeny D, was 11%.

TABLE 3

| Male parent HDF-1 | Female parent B | Progeny C (F1) |
|---|---|---|
| (1) Heliotropium plant | (1) Heliotropium plant | (1) Heliotropium plant |
| having 5 outer petals, 2 | having 5 outer petals | having 5 outer petals, |
| inner petaloids | (2) Length of nodes | 4 inner petaloids (9 |
| (2) Length of nodes | on a stem: short | petals in total) |
| on a stem: medium | (3) Color of the petals: | (2) Length of nodes |
| (3) Color of the petals: | white | on a stem: short |
| purple | (4) Size of the leaves: | (3) Color of the petals: |
| (4) Size of the leaves: | small | white |
| small | | (4) Size of the leaves: |
| | | small |

TABLE 4

| Parents | Progeny D |
|---|---|
| Progeny C in the | (1) Heliotropium plant having 5 |
| above mentioned | outer petals, 5 inner petaloids |
| Table 3 | (2) Length of nodes on a stem: |
| | long |
| | (3) Color of the petals: purple |
| | (4) Size of the leaves: small |

(3) Table 5 shows the crossing result between E as a female and HDF-1 as a male. As a result, we obtained Progeny F. And the ratio of Heliotropium plant, which has more than two inner petaloids among F1 generation, was 1.5%. Furthermore, as shown in Table 6, crossing between Progeny C with each other was conducted. As a result, we obtained Progeny G. And the ratio of Heliotropium plant, which has more than two inner petaloids among Progeny G, was 16%.

TABLE 5

| Male parent HDF-1 | Female parent E | Progeny F (F1) |
|---|---|---|
| (1) Heliotropium plant | (1) Heliotropium plant | (1) Heliotropium plant |
| having 5 outer petals, 2 | having 5 outer petals | having 6 outer petals, |
| inner petaloids | (2) Length of nodes | 4 inner petaloids (10 |
| (2) Length of nodes | on a stem: short | petals in total) |
| on a stem: medium | (3) Size of the petals: | (2) Length of nodes |
| (3) Size of the petals: | large | on a stem: short |
| medium | (4) Size of the leaves: | (3) Size of the petals: |
| (4) Size of the leaves: | small | large |
| medium | | (4) Size of the leaves: |
| | | small |

TABLE 6

| Parents | Progeny G |
|---|---|
| Progeny F in the above | (1) Heliotropium plant having 6 |
| mentioned Table 5 | outer petals, 5 inner petaloids |
| | (2) Length of nodes on a stem: |
| | long |
| | (3) Color of the petals: purple |

As shown in these tables, a common trait among these progeny is having multiple petals only. Other traits are not linked to the multiple petal trait. It means that a Heliotropium plant with various traits, which has a multiple petal gene, can be produced in a proper manner. This indicates the multiple petal gene is not linked to other genes such as a gene of flower color, a gene of storage roots, a gene of leaf color, a gene of length of nodes, a gene of disease resistance and so on. Therefore, this phenotype, which is more than 6 petals, can be introgressed from this line into any desired Heliotropium cultivar.

As mentioned above, the multiple petal trait can be stably and predictably introgressed into diverse Heliotropium genetic backgrounds. The instant Heliotropium can be used as a male or female parent in crosses for introducing the trait into new Heliotropium plants, thereby creating diverse Heliotropium genetic backgrounds. Therefore, a commercially valuable Heliotropium plant can be obtained by introgressing other traits.

### Example 10

<Cross Breeding of the Multiple Petal Gene into Heliotropium Cultivars (Progeny)>

(1) Table 7 shows the result of a cross between the variety “HDF-1” as a female and A1, which was obtained from the cultivar “Marine” by the inventor, as a male. 2% of the progeny A2 (F1 generation) had inner petaloids. And a cross between A2s, which had more than two petaloids, with each other was made. 9% of the progeny A3 (F2 generation) had more than two inner petaloids.

TABLE 7

| | | Progeny | Progeny |
|---|---|---|---|
| Female parent | Male parent A1 | A2 (F1) | A3 (F2) |
| Heliotropium | Commercially | 2% of the | 9% of the |
| arborescens | available | flowers | flowers |
| “HDF-1”having | Heliotropium | having 2 | having 2 |
| 2 inner petaloids | arborescens | inner | inner |
| | “Marine”having | petaloids | petaloids |
| | no petaloid | | |

(2) Table 8 shows the result of a cross between the Heliotropium variety “HDF-1”, having 5 outer petals and 2 inner petaloids as a female, with the Heliotropium variety “A4”, having 5 outer petals as a male, to produce the progeny. The progeny A5 (F1 generation) with 8 petals, consisting of 5 outer petals and 3 inner petaloids, was obtained. The ratio of the progeny A5 having more than two inner petaloids was 2%. And selecting the plants from the progeny A5 (F1 generation) and a cross was made between the selected plants with each other. The progeny A6 (F2 generation) with 6 inner petaloids was obtained. The ratio of the progeny A6 having more than two inner petaloids was 14%. The size of the largest inner petaloid among A6 was more than one-third of the size of the outer petals.

TABLE 8

| | | Progeny | Progeny |
|---|---|---|---|
| Female parent | Male parent A4 | A5 (F1) | A6 (F2) |
| Heliotropium | Commercially | 2% of the | 14% of the |
| arborescens | available | flowers | flowers |
| “HDF-1”having | Heliotropium | having more | having more |
| 5 outer petals | arborescens | than 2 inner | than 2 inner |
| and 2 inner | having 5 outer | petaloids | petaloids |
| petaloids | petals | | |

(3) Table 9 shows the result of a cross between the Heliotropium variety “HDF-1”, having 5 outer petals and 2 inner petaloids as a male, with the Heliotropium variety A16, having 5 outer petals as a female, to produce the progeny. The progeny A16 (F1 generation) did not have a flower with more than two petaloids. However, there were some flowers among the progeny A17 (F2 generation), which was obtained by crossing A16s with each other, having more than two petaloids. The ratio of the progeny A17 having more than two inner petaloids was 2%. As mentioned above, in case when a crossing was made by using the Heliotropium variety “HDF-1” as a male, the ratio of the progeny A16 (F1 generation) having more than two inner petaloids was lower than the ratio of the progeny having more than two inner petaloids, where crossing was made by using the Heliotropium variety “HDF-1” as a female. For these reasons, it has proved that flowers with more than two inner petaloids can surely be obtained at least among F2 generation, in case when the Heliotropium variety “HDF-1” is crossed either one as a female or a male.

TABLE 9

| | | Progeny | Progeny |
|---|---|---|---|
| Male parent | Female parent A15 | A16 (F1) | A17 (F2) |
| Heliotropium | Commercially | No flower | 2% of the |
| arborescens | available | having inner | flowers |
| “HDF-1”having | Heliotropium | petaloids | having |
| 5 outer petals | arborescens | | 2 inner |
| and 2 inner | “Marine”having | | petaloids |
| petaloids | 5 petals | | |

(4) Table 10 shows the result of a cross between the Heliotropium variety “HDF-1”, having 5 outer petals and 2 inner petaloids as a female, with the Heliotropium variety A20, having 5 outer petals as a male, to produce the progeny. The progeny A21, A22 (F1 generation) with 8 petals, consisting of 5 outer petals and 3 inner petaloids, were obtained. The ratio of the progeny (F1 generation) having more than two inner petaloids was 2.5%. And a cross was made between A21 and A22. The ratio of the progeny A23 (F2 generation) having more than two inner petaloids was 11%. Among F2 generation, there was a variety A25 having five inner petaloids, and the size of the largest petaloid of A25 was more than half of the size of the outer petals.

TABLE 10

| | | Progeny | Progeny |
|---|---|---|---|
| Female parent | Male parent A20 | A21and A22 (F1) | A23 (F2) |
| Heliotropium | Commercially | 2.5% of the | 11% of the |
| arborescens | available | flowers | flowers |
| “HDF-1”having | Heliotropium | having more | having more |
| 5 outer petals | plant having | than 2 | than 2 |
| and 2 inner | 5 petals | inner | inner |
| petaloids | | petaloids | petaloids |

Herein, Heliotropium plant originated from tropical highlands, such as mountains, which are cool semi-arid regions, in the Andes in Peru, Colombia, Ecuador (1,000-2,000 meter high). The climate in these regions is stable throughout the year. Thereby, when a Heliotropium plant is placed in a hot, cool, or humid place, it may disturb the flowers vitality. In the worst case scenario, the Heliotropium plant cannot fully express its original flowering physiology.

The inventor crosses about 120,000 plants every year and selects only about 120 plants from the progenies. These 120 plants, which are 0.1% out of 120,000 plants, can be utilized in order to breed new varieties. A plant with a desired characteristic can cross with another plant. In a word, the characteristic of the selected plant is repeatable even if it is in very small amounts. In case when the occurrence ratio is 0.1%, the selected plant could be easily utilized so as to produce new varieties. Over 1% of the occurrence rate is a very high rate so that the plant can be easily utilized to produce new varieties.

As mentioned above, the Heliotropium having a multiple petal gene can be used as a male or female parent in crosses for introducing the trait into new Heliotropium plants. However, the multiple petal gene shows the characteristics of neither a dominant gene nor a recessive gene. 1-10% of the progeny exhibit the instant trait. In other words, the multiple petal gene is thought to be neither a dominant gene nor a recessive gene, and is not in accordance with the Mendel's law. Needless to say, there are many other traits, such as the color of the flower, having an incomplete heredity.

### Embodiment 2

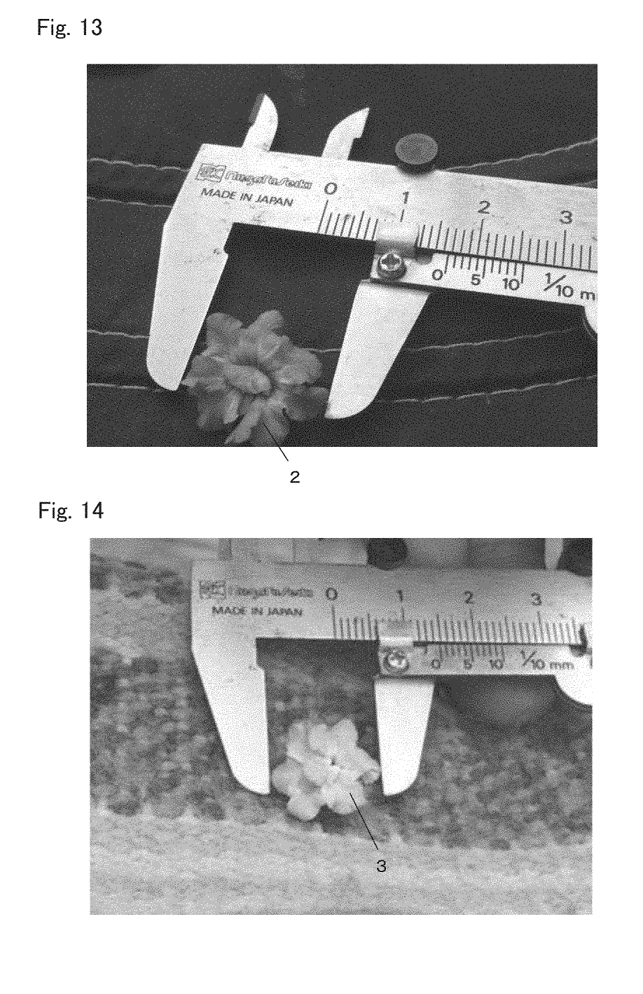

Referring from FIG. 13 to FIG. 16, the Heliotropium plant according to Embodiment 2 will be described.

The following characteristics have been represented and observed at the Heliotropium plant according to Embodiment 2 of the present invention. The Heliotropium plant described herein has the characteristics that the diameter of the flowers is more than 12.5 mm, preferably more than 15 mm in addition to having more than 6 petals as a double-flowers type. On the other hand, conventional Heliotropium species usually have flowers whose size of the diameter is around 3 mm to 8 mm at best. Therefore, it can be said that the Heliotropium plant with double flowers, such as having more than 6 petals, and large-sized flowers is a new cultivar with completely new characteristics. These characteristics and the combination of these characteristics are clearly distinguished as a new Heliotropium variety from the conventional Heliotropium varieties.

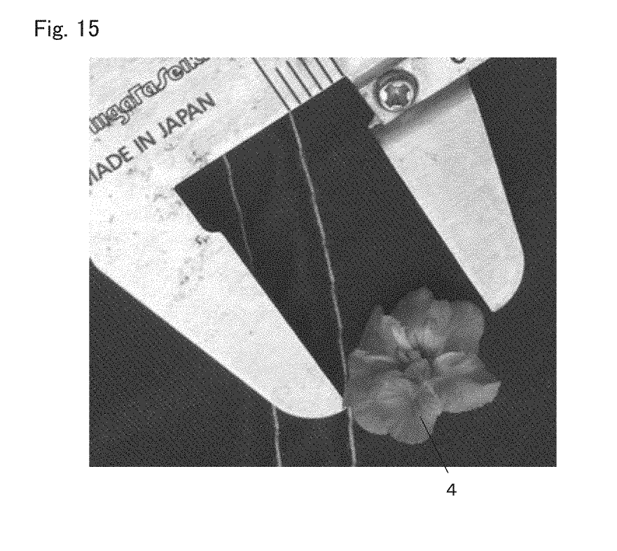

For example, the double-flower type Heliotropium 2 with 7 to 13 petals, whose flower diameter is about 16.5 mm, is shown in FIG. 13. The double-flower type Heliotropium 3 with 7 to 13 petals, whose flower diameter is about 15 mm, is shown in FIG. 14. And the double-flower type Heliotropium 4 with 7 to 13 petals, whose flower diameter is about 15 mm, is shown in FIG. 15.

As mentioned above, the Heliotropium plant has not only more than 6 petals, which are more than those of conventional Heliotropium cultivars, but also much large-sized flowers than those of conventional Heliotropium cultivars. As a result, this new Heliotropium plant has more commercial value.

It is to be understood that the Heliotropium plant described herein is not intended to limiting the scope of morphological and ecological characteristics of Heliotropium, except for the number of petals and the size of the flowers.

Generally speaking, the variety Heliotropium.europaeum is famous for the large size of its flowers and it is sometimes called as big Heliotropium. However, Heliotropium.europaeum is very different from Heliotropium.arborescens in terms of its traits. Specifically, Heliotropium.arborescens is what we call a tree that can live for a long time. On the other hand, Heliotropium.europaeum is a grass and weak to cold. The flower diameter of Heliotropium.europaeum is large and sometimes reaches around 10 mm. However, there is no Heliotropium.europaeum flower whose diameter is more than 12.5 mm. It has not been observed that the flower diameter of Heliotropium.europaeum could reach more than 15 mm. What is more important, Heliotropium.europaeum cannot cross with Heliotropium.arborescens. There has been no successful crossing between Heliotropium.europaeum and Heliotropium.arborescens. In other words, there is no proof that the flowers of Heliotropium.arborescens can be larger by making use of Heliotropium.europaeum. Therefore, it is noted that the Heliotropium plant according to Embodiment 2 does not utilize any gene from Heliotropium.europaeum.

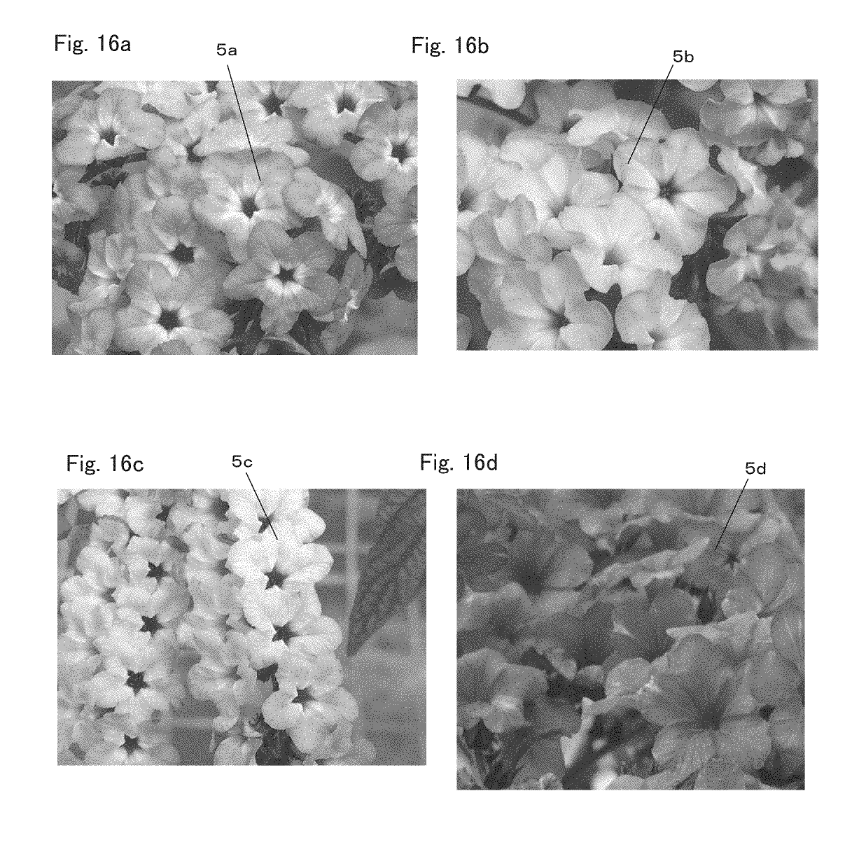

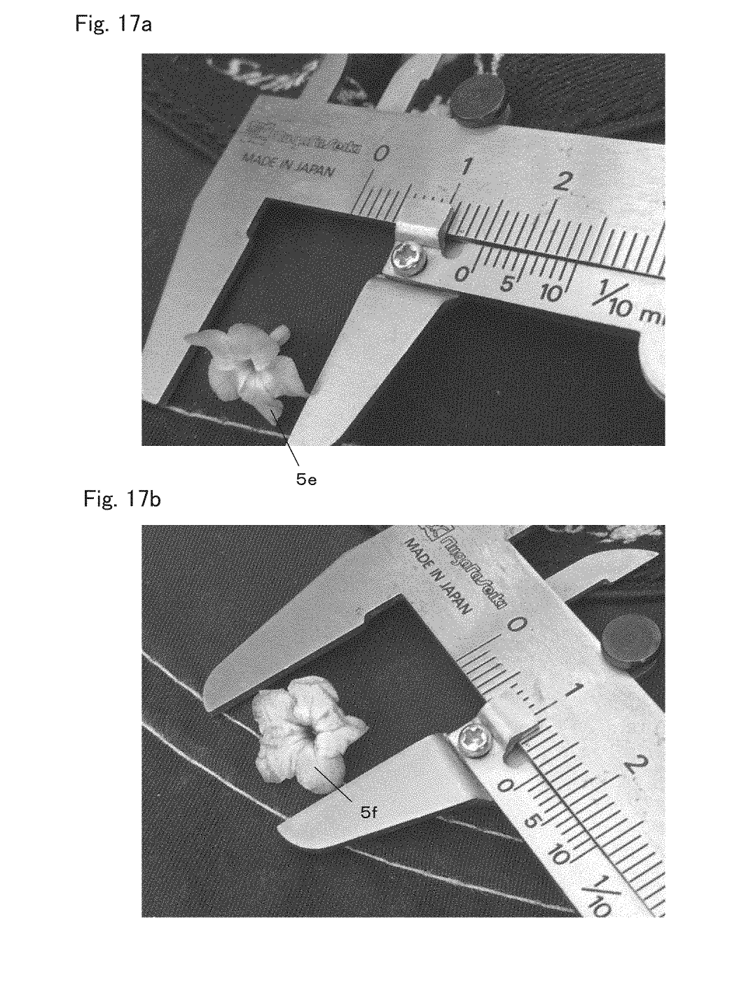

For references, in FIG. 16 and FIG. 17, Heliotropium 5a to 5f have a flower diameter of more than 12.5 mm, preferably 15 mm, and have flowers with 5 petals. In particular, the flower diameter of Heliotropium 5e and 5f are more than 15 mm in FIG. 17. As shown in these figures, the inventor has also succeeded in creating Heliotropium whose flower diameter is larger than before, while the number of the flowers is 5 as same as the conventional Heliotropium cultivars. Needless to say, the only difference between Heliotropium 5a to 5f and Heliotropium 2 to 4 is the number of the petals according to Embodiment 2, all other traits such as breeding method and producing method, which are written in this specification, are applicable to both of them.

### Embodiment 3

The Heliotropium plant according to Embodiment 3 will be described. The following characteristics have been represented and observed in the Heliotropium plant according to Embodiment 3 of the present invention. The Heliotropium plant described herein has the characteristics of not having stamens with anther in addition to having double flowers with more than 6 petals such as the Heliotropium 1 shown in FIG. 7. On the other hand, conventional Heliotropium species usually have 5 stamens. Therefore, it can be said that the Heliotropium plant according to Embodiment 3, with characteristics of having double flowers with more than 6 petals and not having stamens with anther, is a new cultivar with completely new characteristics. These characteristics and the combination of these characteristics are clearly distinguished as a new Heliotropium variety from the conventional Heliotropium varieties.

The Heliotropium plant according to Embodiment 3 has not only more commercial values by having more petals, but also has no stamen with anther. Since the Heliotropium plant is a male sterile plant, which can avoid producing seeds, it has more number of blooms. As a result of this, it is clear that the Heliotropium plant according to Embodiment 3 has more commercial value.

It is to be understood that the Heliotropium plant described herein is not intended to limiting the scope of morphological and ecological characteristics of Heliotropium, except for the number of petals and stamen.

### Example

Next, an example of the Heliotropium plant according to the Embodiment 3 of the present invention will be explained as follows. However, it is noted that the form of the present invention is not limited to the described practical example.

Similar to practical examples described in Embodiment 1, the selection procedure is as follows: (1) crossing above mentioned mutant individual of Heliotropium, which has more petals than its parents. (2) Next, collecting seeds, and selecting Heliotropium cultivars having even more petals obtained by breeding the seeds. (3) And crossing the said Heliotropium cultivars, and then selecting Heliotropium cultivars having more petals (repeating the crossing and selection) for multiple generations in order to gradually enhance the characteristic of having more petals. In this example, the Heliotropium cultivars with characteristics of not having (or having a fewer) stamens with anther in addition to having more petals were selected.

Because this Heliotropium plant is a male sterile plant that has no stamen and no anther, it has been observed that the seeds are not likely to be produced unless otherwise a non-self pollen has been deposited from another Heliotropium plant.

It is to be noted that the Heliotropium plant can be proliferated (asexually reproduced) with herbaceous cutting and other techniques. Herbaceous cutting can be conducted with known methods for a person having ordinary skill in the art. For example, after cutting a tissue from the Heliotropium paint, the tissue has been cultivated under optimum environments in order to produce a plantlet having roots and shoots. And then, the plantlet is raised under optimum environments. The tissue can also be cultivated through mericloning, which extracts approximately 1 mm of growing point from sprout and cultivates it in sterile culture.

As described above, the Heliotropium plant according to Embodiment 3 can have more petals than conventional Heliotropium plants. Therefore, the Heliotropium plant has more petals and the number of blooms per plant increases, because the Heliotropium plant is a male sterile plant, and its appearance becomes more compact. Hence, these characteristics can enhance the commercial value of the Heliotropium plant.

The present invention is not limited to the configuration of the above Embodiments and various modifications can be made within a scope not changing the gist of the present invention. Generally speaking, plants can express their original traits under a good condition, on the other hand, plants cannot express their original traits under a harsh condition. Therefore, if a Heliotropium plant was raised in a bad condition, the number of the petals would decrease. For example, not every flower is a double-flower type with many petals. In some cases, 80% of the flowers have more than 12 petals, while 20% of the flowers have less than 12 petals. It is true that every flower can show its original traits under a good condition. In other words, a flower can recover its original trait under a good condition, even if it was placed in a harsh condition for a while. This theory is not only in the case for Heliotropium plants, but for all plants in general.

Furthermore, it can be expected that genes in the DNA and RNA relating to the characteristics of the flowering physiology, having no stamens or a female sterility, will be extracted from the abovementioned Heliotropium plants and embedded the genes into other cells. This will lead to reproducing other plants acquiring such DNA and RNA, and to creating plants having these characteristics other than Heliotropium.

What is claimed is:

1. A Heliotropium.arborescens plant comprising at least one flower with more than 6 petals,
wherein the Heliotropium.arborescens plant comprises a multiple petal gene, as found in Heliotropium.arborescens variety “HDF-1”, representative tissue culture having been deposited with the international depositary authority under the deposit number FERM BP-22339.

2. The Heliotropium.arborescens plant of claim 1, wherein the flower includes 7 to 10 petals.

3. The Heliotropium.arborescens plant of claim 2, wherein the flower includes 11 to 13 petals.

4. The Heliotropium.arborescens plant of claim 3, wherein the flower includes more than 14 petals.

5. The Heliotropium.arborescens plant of claim 1,
wherein the flower of the Heliotropium plant is a double-flower type with outer petals as a gamopetalous flower, and inner petaloids, which are separated with each other, near the center of the flower,
and wherein “the number of petals” consists of the number of the outer petals and the inner petaloids of the Heliotropium.

6. The Heliotropium.arborescens plant of claim 5, wherein the number of the inner petaloids is more than 1 petal.

7. The Heliotropium.arborescens plant of claim 5, wherein the number of the inner petaloids is more than 4 petals.

8. The Heliotropium.arborescens plant of claim 5, wherein the number of the outer petals is more than 7 petals.

9. The Heliotropium.arborescens plant of claim 5,
wherein the size of the separated inner petaloids is more than half of the size of the outer petals.

10. The Heliotropium.arborescens plant of claim 5,
wherein the size of the separated inner petaloids is more than ⅔ of the size of the outer petals.

11. The Heliotropium.arborescens plant of claim 1,
wherein the Heliotropium.arborescens plant comprises no pollen.

12. The Heliotropium.arborescens plant of claim 11,
wherein the Heliotropium.arborescens plant comprises no stamen.

13. The Heliotropium.arborescens plant of claim 1,
wherein the Heliotropium.arborescens plant is produced from the tissue culture having the deposit number FERM BP-22339, which is the variety “HDF-1”.

14. Seed of the Heliotropium.arborescens of claim 1.

15. A tissue culture of cells produced from the plant of claim 1,
wherein said cells of the tissue culture are produced from a plant part selected from the group consisting of seed, leaf, pollen, embryo, cotyledon, hypocotyl, meristematic cell, root, root tip, pistil, anther, flower, stem, and petiole.

16. A method of producing the Heliotropium.arborescens plant of claim 1 comprising;
(a) crosssing, as a male or female parent, the Heliotropium.arborescens plant of claim 1 that has more than 6 petals, with a Heliotropium.arborescens plant that does not have more than 6 petals,
(b) selecting progeny that has more than 6 petals.

17. A Heliotropium.arborescens plant obtained by utilizing either one of pollen, ovum, cell, and genetic information relating to DNA and RNA of the Heliotropium.arborescens plant of claim 1.

18. A method for producing a Heliotropium.arborescens plant comprising;
(a) utilizing either one of pollen, ovum, cell, and genetic information relating to DNA and RNA of the Heliotropium.arborescens plant of claim 1 so as to produce the Heliotropium.arborescens plant.
